(19) United States
(12) Patent Application Publication
(10) Pub. No.: US 2025/0281083 A1
(43) Pub. Date: Sep. 11, 2025

(54) OXYGEN SATURATION MEASUREMENT GUIDE METHOD AND WEARABLE DEVICE THEREFOR

(71) Applicant: SAMSUNG ELECTRONICS CO., LTD., Suwon-si (KR)

(72) Inventors: Hyunjun JUNG, Suwon-si (KR); Jinho KIM, Suwon-si (KR); Gunwoo JIN, Suwon-si (KR)

(21) Appl. No.: 19/214,626

(22) Filed: May 21, 2025

Related U.S. Application Data
(63) Continuation of application No. PCT/KR2023/016999, filed on Oct. 30, 2023.

(30) Foreign Application Priority Data
Nov. 25, 2022 (KR) 10-2022-0160309
Jan. 10, 2023 (KR) 10-2023-0003265

(51) Int. Cl. A61B 5/1455; A61B 5/00; A61B 5/11; G16H 40/63

(52) U.S. Cl. CPC A61B 5/14552; A61B 5/1125; A61B 5/7264; A61B 5/742; A61B 5/746; A61B 5/7475; G16H 40/63; A61B 2560/0462; A61B 2562/0219

(57) ABSTRACT

An embodiment may include: an inertial sensor; a PPG sensor; a display module; a processor; and a memory connected electrically to the processor and configured to store instructions executable by the processor, wherein the instructions, when executed by the processor, cause the wearable device to: in response to a request for measurement of oxygen saturation, acquire motion data from the inertial sensor; responsive to a determination on the basis of the motion data that there is no motion in a wearable device, determine a wearing hand on which the wearable device is worn, on the basis of the motion data; detect a tilt of the wearable device while measuring the oxygen saturation by means of the PPG sensor; distinguish between wearing positions of the wearable device on the basis of the wearing hand and the tilt; and provide different user interfaces according to the wearing positions.

### BACKGROUND

An embodiment of the disclosure relates to an oxygen saturation measurement guide method and a wearable device therefor.

In accordance with development of digital technologies, various types of electronic devices, such as a mobile terminal, a personal digital assistant (PDA), an electronic notebook, a smartphone, a tablet personal computer (PC), or a wearable device, have been widely used. Electronic devices have been continuously improved in terms of hardware and/or software of the electronic devices to support and increase functions thereof.

For example, an electronic device like a wearable device may be provided in various forms, such as a smartwatch, smart glasses, and smart bands that may come in contact with (or worn on) the user's body. A wearable device may collect and analyze various information (e.g., biometric or activity information) associated with a user so as to provide various functions (e.g., exercise information or health information) to the user.

For example, a wearable device may measure oxygen saturation using a pulse oximetry method. Oxygen saturation may an indicator used in a vital check along with electrocardiogram, blood pressure, pulse, respiratory rate, and body temperature. A pulse oximetry method may be a method of measuring oxygen saturation using the ratio of the absorbance of increased blood flow at two wavelengths (e.g., RED, Infrared) using temporary volume changes in arterial blood caused by cardiac output.

### SUMMARY

According to an embodiment of the disclosure, a wearable device may include an inertial sensor, a PPG sensor, a display module, a processor operatively coupled to at least one of the inertial sensor, the PPG sensor, and the display module, and a memory connected electrically to the processor and configured to store instructions executable by the processor, wherein the instructions, when executed by the processor, cause the wearable device to, in response to a request for measurement of oxygen saturation, acquire motion data from the inertial sensor, responsive to a determination, based on the motion data, that there is no motion of the wearable device, determine a wearing hand on which the wearable device is worn, based on the motion data, detect a tilt of the wearable device during the measurement of oxygen saturation by means of the PPG sensor, classify wearing positions of the wearable device, based on the wearing hand and the tilt, and provide different user interfaces according to the wearing positions.

According to an embodiment, an operation method of a wearable device may include an operation of, in response to a request for measurement of oxygen saturation, acquiring motion data from an inertial sensor included in the wearable device, an operation of, if it is determined, based on the motion data, that there is no motion of a wearable device, determining a wearing hand on which the wearable device is worn, based on the motion data, an operation of detecting a tilt of the wearable device during the measurement of oxygen saturation by means of a PPG sensor included in the wearable device, an operation of classifying wearing positions of the wearable, device based on the wearing hand and the tilt, and an operation of providing different user interfaces according to the wearing positions.

### BRIEF DESCRIPTION OF THE DRAWINGS

FIG. 1 is a block view illustrating an electronic device in a network environment according to an embodiment.

FIG. 2 is a front perspective view illustrating a wearable device according to various embodiments.

FIG. 3 is a rear perspective view illustrating a wearable device according to various embodiments.

FIG. 4 is a flowchart illustrating an operation method of a wearable device according to an embodiment.

FIGS. 5A to 5D are views illustrating an example of a user interface provided in a wearable device according to an embodiment.

FIG. 6 is a flowchart illustrating a method of classifying wearing positions according to a wearing hand in a wearable device according to an embodiment.

FIGS. 7A and 7B are views illustrating an example of classifying wearing positions according to a wearing hand in a wearable device according to an embodiment.

FIG. 8 is a flowchart illustrating a method of providing a user interface depending on wearing suitability in a wearable device according to an embodiment.

FIGS. 9A and 9B are views illustrating an example of classifying wearing suitability of a wearable device according to an embodiment.

FIG. 10 is a view illustrating an example of providing a guide associated with oxygen saturation measurement in a wearable device according to an embodiment.

### DETAILED DESCRIPTION

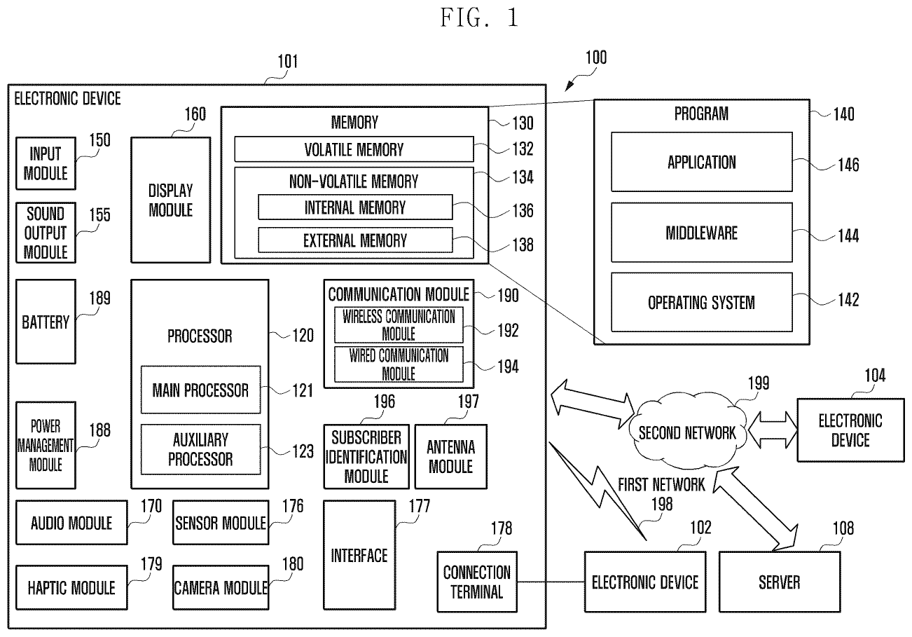

FIG. 1 is a block diagram illustrating an electronic device 101 in a network environment 100 according to certain embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™ wireless-fidelity (Wi-Fi) direct, or infrared data association (IrDA)) or the second network 199 (e.g., a long-range communication network, such as a legacy cellular network, a 5th generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4th generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to certain embodiments, the antenna module 197 may form a mmWave antenna module. According to an embodiment, the mmWave antenna module may include a printed circuit board, an RFIC disposed on a first surface (e.g., the bottom surface) of the PCB, or adjacent to the first surface and capable of supporting a designated high-frequency band (e.g., the mmWave band), and a plurality of antennas (e.g., array antennas) disposed on a second surface (e.g., the top or a side surface) of the PCB, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various embodiments disclosed herein may be one of various types of electronic devices. The electronic devices may include, for example, a portable communication device (e.g., a smart phone), a computer device, a portable multimedia device, a portable medical device, a camera, a wearable device, or a home appliance. The electronic device according to embodiments of the disclosure is not limited to those described above.

It should be appreciated that various embodiments of the disclosure and the terms used therein are not intended to limit the technological features set forth herein to particular embodiments and include various changes, equivalents, or alternatives for a corresponding embodiment. With regard to the description of the drawings, similar reference numerals may be used to designate similar or relevant elements. A singular form of a noun corresponding to an item may include one or more of the items, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “a first”, “a second”, “the first”, and “the second” may be used to simply distinguish a corresponding element from another, and does not limit the elements in other aspect (e.g., importance or order). It is to be understood that if an element (e.g., a first element) is referred to, with or without the term “operatively” or “communicatively”, as “coupled with/to” or “connected with/to” another element (e.g., a second element), it means that the element may be coupled/connected with/to the other element directly (e.g., wiredly), wirelessly, or via a third element.

As used herein, the term “module” may include a unit implemented in hardware, software, or firmware, and may be interchangeably used with other terms, for example, “logic,” “logic block,” “component,” or “circuit”. The “module” may be a minimum unit of a single integrated component adapted to perform one or more functions, or a part thereof. For example, according to an embodiment, the “module” may be implemented in the form of an application-specific integrated circuit (ASIC).

Various embodiments as set forth herein may be implemented as software (e.g., the program 140) including one or more instructions that are stored in a storage medium (e.g., the internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each element (e.g., a module or a program) of the above-described elements may include a single entity or multiple entities, and some of the multiple entities mat be separately disposed in any other element. According to various embodiments, one or more of the above-described elements may be omitted, or one or more other elements may be added. Alternatively or additionally, a plurality of elements (e.g., modules or programs) may be integrated into a single element. In such a case, according to various embodiments, the integrated element may still perform one or more functions of each of the plurality of elements in the same or similar manner as they are performed by a corresponding one of the plurality of elements before the integration. According to various embodiments, operations performed by the module, the program, or another element may be carried out sequentially, in parallel, repeatedly, or heuristically, or one or more of the operations may be executed in a different order or omitted, or one or more other operations may be added.

To improve the accuracy of pulse oximetry, methods such as applying pressure to reduce venous blood components, guiding the user to change the measurement position (e.g., below or above the elbow), and using signal processing techniques to remove noise may be used. However, it may be difficult to apply the methods above to a wearable device.

An embodiment may provide a method and a device which determine a wearing position by means of an inertial sensor of a wearable device and provide a user guide corresponding to the determined wearing position or determine a quality of oxygen saturation measured through a photoplethysmography (PPG) sensor and provide a user guide corresponding to the determined quality of oxygen saturation.

According to an embodiment, by determining a wearing position by means of an inertial sensor of a wearable device and providing a user guide corresponding to the determined wearing position, the user may be guided to a precise position in which the user wears the wearable device to measure oxygen saturation.

According to an embodiment, by determining the quality of oxygen saturation measured through a PPG sensor and providing a user guide corresponding to the determined quality of the oxygen saturation, the accuracy of the oxygen saturation measurement may be improved.

According to an embodiment, by providing different user guides depending on the wearing position or the quality of oxygen saturation, the user may follow the guides to improve a wearing state of the wearable device and increase the usability of the wearable device.

According to an embodiment, by providing gradually different guides based on motion detection or the quality of oxygen saturation, each user may be guided to an optimized measurement position to increase the success rate of the measurement.

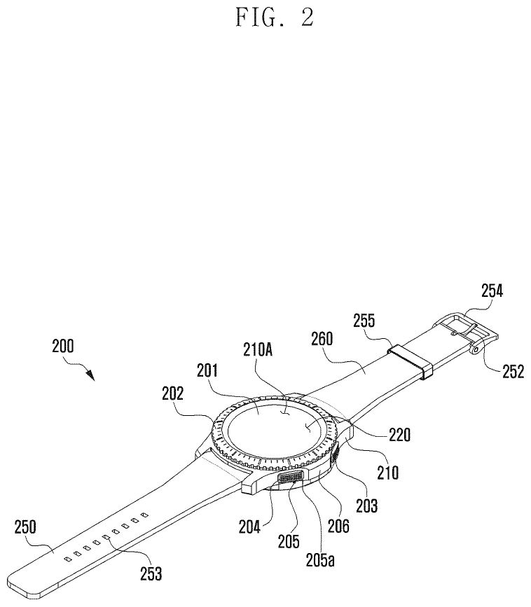

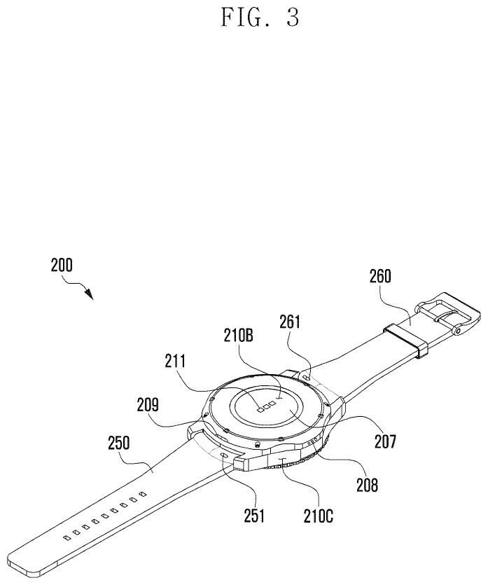

FIG. 2 is a front perspective view illustrating a wearable device according to various embodiments, and FIG. 3 is a rear perspective view illustrating a wearable device according to various embodiments.

Referring to FIGS. 2 and 3, the wearable device (e.g., the electronic device 101 in FIG. 1) according to various embodiments may include a housing 210 including a first surface (or front surface) 210A, a second surface (or rear surface) 210B, and a lateral surface 210C surrounding a space between the first surface 210A and the second surface 210B, and a coupling member 250 or 260 connected to at least a portion of the housing 210 and configured to detachably couple the wearable device 200 to the body (e.g., a wrist, an ankle of the like) of a user. According to another embodiment (not shown), the housing may refer to a structure for configuring a portion of the first surface 210A, the second surface 210B, and the lateral surface 210C in FIG. 2.

According to an embodiment, at least a portion of the first surface 210A may be made of a substantially transparent front plate 201 (e.g., a glass plate including various coating layers or polymer plate). The second surface 210B may be made of a substantially opaque rear plate 207. The rear plate 207 may include, for example, coated or colored glass, ceramic, polymers, metals (e.g., aluminum, stainless steel (STS), or magnesium), or a combination of at least two thereof. The lateral surface 210C may be coupled to the front plate 201 and the rear plate 207 and made of a lateral bezel structure (or “lateral member”) 206 including a metal and/or polymer. In an embodiment, the rear plate 207 and the lateral bezel structure 206 may be integrally configured and include an identical material (e.g., a metal material such as aluminum). The coupling member 250 or 260 may be made of various materials and in various shapes. The coupling member may be configured to be integrally and to multiple unit links to be movable with each other by using a woven fabric, leather, rubber, urethane, metal, ceramic, or a combination of at least two of the materials.

According to various embodiments, the wearable device 200 may include at least one of a display 220 (e.g., the display module 160 in FIG. 1), an audio module 205 or 208, a sensor module 211 (e.g., the sensor module 176 in FIG. 1), a key input device 202, 203, or 204 (e.g., the input module 150 in FIG. 1), and a connector hole 209. In an embodiment, the wearable device 200 may omit at least one of the components (e.g., the key input device 202, 203, or 204, the connector hole 209, or the sensor module 211) or additionally include another component.

The display 220 may be exposed to outside through, for example, a substantial portion of the front plate 201. The display 220 may have a shape corresponding to the shape of the front plate 201 and have various shapes such as a circle, an oval, or a polygon. The display 220 may be combined to or disposed adjacent to a touch sensing circuit, a pressure sensor for measuring a strength (pressure) of a touch, and/or a fingerprint sensor.

The audio module 205 or 208 may include a microphone hole 205a and a speaker hole 208. A microphone configured to acquire a sound from outside may be disposed in the microphone hole 205a and in an embodiment, multiple microphones may be arranged to detect a direction of a sound. The speaker hole 208 may be used for an outer speaker and a receiver for calling.

The sensor module 211 may generate an electrical signal or a data value corresponding to an internal operation state or external environment state of the wearable device 200. The sensor module 211 may include, for example, a biosensor module 211 (e.g., a HRM sensor) disposed on the second surface 210B of the housing 210. The wearable device 200 may further include at least one sensor module not shown in the drawings, for example, a gesture sensor, a gyro sensor, an air pressure sensor, a magnetic sensor, an acceleration sensor, a grip sensor, a color sensor, an infrared (IR) sensor, a biometric sensor, a temperature sensor, humidity sensor, or an illuminance sensor.

The key input device 202, 203, or 204 may include a wheel key 202 disposed on the first surface 210A of the housing 210 and rotatable in at least one direction, and/or a side button key 202 or 203 disposed at the lateral surface 210C of the housing 210. The wheel key may have a shape corresponding to the front plate 202. In another embodiment, the wearable device 200 may not include a portion or entirety of the key input device 202, 203, or 204, and the excluded key input device 202, 203, or 204 may be implemented in various forms such as a soft key on the display 220. The connector hole 209 may include another connector hole (not shown) capable of receiving a connector (e.g., a USB connector) configured to transmit or receive power and/or data to or from an external electronic device and a connector for transmitting or receiving an audio signal to or from an external electronic device. The wearable device 200 may further include, for example, a connector cover (not shown) configured to cover a portion of the connector hole 209 and block the ingress of external foreign substances into the connector hole.

The coupling member 250 or 260 may be detachably coupled to at least a portion of the housing 210 by using a locking member 251 and 261. The coupling member 250 or 260 may include one or more of a fixation member 252, a fixation member fastening hole 253, a band guide member 254, and a band fixation ring 255.

The fixation member 252 may be configured to fix the coupling member 250 or 260 and the housing 210 to a body portion (e.g.: wrist and ankle) of a user. The fixation member fastening hole 253 may fix the coupling member 250 or 260 and the housing 210 to a body portion of a user by counteracting with the fixation member 252. The band guide member 254 is configured to limit the motion range of the fixation member 252 when the fixation member 252 is fastened to the fixation member fastening hole 253 so that the coupling member 250 or 260 is closely bound to a body portion of a user. The band fixation ring 255 may limit the motion range of the coupling member 250 or 260 in a state where the fixation member 252 is fastened to the fixation member fastening hole 253.

According to an embodiment of the disclosure, a wearable device 200 may include an inertial sensor (e.g., sensor module 176), a PPG sensor (e.g., sensor module 176), a display module 220, a memory 130, and a processor 120 operatively connected to at least one of the inertial sensor, the PPG sensor, the display module, or the memory, wherein the processor may be configured to, in response to a request for measurement of oxygen saturation, acquire motion data from the inertial sensor, when it is determined, based on the motion data, that there is no motion of the wearable device, determine a wearing hand on which the wearable device is worn, detect a tilt of the wearable device while measuring the oxygen saturation by means of the PPG sensor, classify wearing positions of the wearable device based on the wearing hand and the tilt, and provide different user interfaces according to the wearing positions.

The processor may be configured to, in case that the wearing hand corresponds to the left hand, apply a left-hand tilt detection algorithm and, in case that the wearing hand corresponds to the right hand, apply a right-hand tilt detection algorithm.

The processor may be configured to determine whether an angle between a wrist direction of the wearable device and the ground exceeds a first reference value according to the left-hand tilt detection algorithm and classify the wearing position of the wearable device according to a result of the determination.

The processor may be configured to determine whether the angle between the wrist direction of the wearable device and the ground is less than a second reference value according to the right-hand tilt detection algorithm and classify the wearing position of the wearable device according to a result of the determination.

The processor may be configured to, in case that the wearing position corresponds to a first position, provide a first user interface associated with the first position, in case that the wearing position corresponds to a second position, provide a second user interface associated with the second position, and in case that the wearing position corresponds to a third position, provide a third user interface associated with the third position, wherein the first position corresponds to a case where the angle between the wrist direction of the wearable device and the ground has a value exceeding a first reference value or less than a second reference value, the second position corresponds to a case where the angle between the wrist direction of the wearable device and the ground has a value between the first reference value and the second reference value, and the third position corresponds to a case where the angle between the wrist direction of the wearable device and the ground has a value opposite to the first position.

The processor may be configured to, in case that the wearing position corresponds to the first position or the second position, maintain oxygen saturation measurement and, in case that the wearing position corresponds to the third position, stop oxygen saturation measurement.

The processor may be configured to acquire an amplitude of alternating current (AC) and an amplitude of direct current (DC) from a PPG signal acquired from the PPG sensor, in case that the amplitude of the AC exceeds the amplitude of the DC, determine that a wearing state of the wearable device is suitable, and in case that the amplitude of the AC is equal to or less than the amplitude of the DC, determine that the wearing state of the wearable device is unsuitable.

The processor may be configured to, when it is determined that the wearing state of the wearable device is suitable, maintain oxygen saturation measurement.

The processor may be configured to determine whether the measured oxygen saturation exceeds a reference value, in case that the measured oxygen saturation exceeds the reference value, provide an oxygen saturation measurement value, and in case that the measured oxygen saturation is equal to or less than the reference value, provide the oxygen saturation measurement value and a warning notification.

The processor may be configured to, when it is determined that the wearing state of the wearable device is unsuitable, stop oxygen saturation measurement.

The processor may be configured to, in case that the number of oxygen saturation measurement stops corresponds to a designated number of times, provide a first measurement guide, and in case that the number of oxygen saturation measurement stops does not correspond to the designated number of times, provide a second measurement guide.

The processor may be configured to determine whether there is no motion of the wearable device after providing the first measurement guide or the second measurement guide.

The processor may be configured to, after the oxygen saturation measurement is requested, in case that the number of detections of the motion of the wearable device corresponds to a designated number of detections, provide a first guide, and after the oxygen saturation measurement is requested, in case that the number of detections of the motion of the wearable device does not correspond to the designated number of detections, provide a second guide.

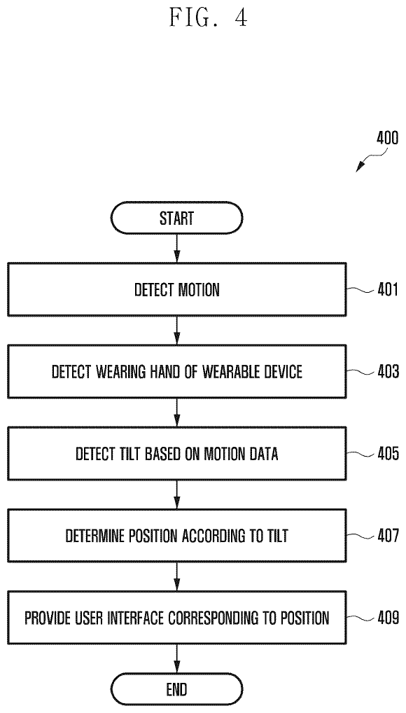

FIG. 4 is a flowchart 400 illustrating an operation method of a wearable device according to an embodiment.

Referring to FIG. 4, in operation 401, a processor (e.g., the processor 120 in FIG. 1) of the wearable device (e.g., the electronic device 101 in FIG. 1 or the wearable device 200 in FIG. 2) according to an embodiment may detect a motion. For example, the processor 120 may detect whether there is a motion of the wearable device 200 by using a sensor module (e.g., the sensor module 176 in FIG. 1) included in the wearable device 200. For example, the sensor module 176 may correspond to an inertial sensor, and the inertial sensor may include an acceleration sensor and a gyro sensor. According to one or more embodiments, the inertial sensor is always operated and thus the processor 120 may acquire motion data from the inertial sensor in real time. The processor 120 may, in case that a measurement of oxygen saturation (e.g., SpO2) is requested, detect whether there is a motion of the wearable device 200. In case that a motion is detected when measuring oxygen saturation, it may cause problems with the oxygen saturation measurement. The processor 120 may, in case that a motion is not detected from the wearable device 200, start oxygen saturation measurement in order to prevent mismeasurement of oxygen saturation.

According to an embodiment, the processor 120 may convert the acquired motion data (e.g., a sensing value of x, y, and z) into an Euler angle, and detect a motion of the wearable device 200 through whether a change (e.g., a motion of 5 degrees or more in 3 seconds) in the converted Euler angle (e.g., one of roll, pitch, or yaw) greater than or equal to a determined threshold is detected during a determined time period. Alternatively, the processor 120 may calculate the amount of change in x, y, and z by using the value of the acquired motion data itself, and detect a motion of the wearable device 200 through whether the calculated amount of change dictates the detection of change greater than or equal to a determined threshold. There are various methods to detect the motion of the wearable device 200, and the processor 120 may detect the motion by using various known calculation methods.

In operation 403, the processor 120 may detect (or determine) a wearing hand of the wearable device 200. The processor 120 may, in case that the motion of the wearable device 200 has not been detected, detect a wearing hand of the wearable device 200, based on the motion data acquired during a process in which the user is wearing the wearable device 200 and raises a hand for measuring oxygen saturation. Alternatively, the processor 120 may provide a user interface for querying the user whether the wearable device 200 is worn on the left or right hand and acquire information about the wearing hand from the user. Alternatively, the processor 120 may acquire information about the wearing hand of the wearable device 200 from configuration information of the wearable device 200. The configuration information may be acquired by directly inputting information about the wearing hand of the wearable device 200.

In operation 405, the processor 120 may detect a tilt, based on the motion data. The processor 120 may, based on the converted Euler angle, detect the tilt of the wearable device 200. The reason for detecting the wearing hand of the wearable device 200 first, and then detecting the tilt of the wearable device 200, may be that the direction of the wearable device 200 alone does not indicate whether a current position of the wearable device 200 is higher or lower than the user's heart. For example, this may be to distinguish when the user is wearing the wearable device 200 on the left wrist and is raising the left hand, and when the user is wearing the wearable device 200 on the right wrist and is lowering the right wrist.

According to an embodiment, the processor 120 may, in case that the wearing hand has been determined, start oxygen saturation measurement by means of a photoplethysmography (PPG) sensor (e.g., the sensor module 176 in FIG. 1). Detecting the wearing hand may be determined before the oxygen saturation measurement. The sensor module 176 may further include a PPG sensor. The PPG sensor may include a light-emitting unit (e.g., an LED (e.g., RED or infrared)), a light-receiving unit (e.g., a photo diode (PD)), an optical structure configured to acquire reflective PPG, and a signal processing unit configured to process the acquired PPG signal. For example, the light-emitting unit and the light-receiving unit of the PPG sensor may be configured as an array of LEDs and PDs having multiple wavelengths, and a spectrometer light source outputting a multi-wavelength laser may be used. The signal processing unit may calculate oxygen saturation (or an oxygen saturation value) by using conventionally well-known method (e.g., measuring an amount of light output from the light-emitting unit that transmits through the user's body (e.g., a finger or wrist) or is reflected and reaches the light-receiving unit). For example, the signal processing unit may calculate oxygen saturation by using a ratio of AC to DC components for each wavelength included in the PPG signal.

For example, the DC component is derived from substances that produce a constant reflection, such as skin, muscle and bone, and venous blood. In case that the human body is stationary and has less motion, the AC component is made mainly of light reflected from the pulsation of arterial blood. The AC component may vary depending on the heart rate and arterial thickness. More light may be reflected or transmitted during systole than during diastole. During systole, arterial blood pressure may increase because the heart is pumping out blood. Increased blood pressure may cause arteries to expand and increase blood flow in the arteries, and increased blood flow may increase light absorption. During diastole, the blood pressure may decrease, and light absorption may also decrease. The processor 120 may apply a different tilt detection algorithm depending on the wearing hand of the wearable device 200 while measuring oxygen saturation.

In case that the wearing hand of the wearable device 200 is the left hand, the pitch, which is a measure of the degree to which the angle between the wrist direction of the wearable device 200 and the ground is tilted, may have a determined positive value (e.g., θ>60°, exceeding a first threshold). On the contrary, in case that the wearing hand of the wearable device 200 is the right hand, the pitch, which is a measure of the degree to which the angle between the wrist direction of the wearable device 200 and the ground is tilted, may have a determined negative value (e.g., θ<−60°, less than a second threshold). The processor 120 may apply a different tilt detection algorithm depending on the wearing hand of the wearable device 200 so as to determine a wearing position of the wearable device 200. The processor 120 may apply a left-hand tilt detection algorithm in case that the wearing hand of the wearable device 200 is the left hand. The processor 120 may apply a right-hand tilt detection algorithm in case that the wearing hand of the wearable device 200 is the right hand.

In operation 407, the processor 120 may determine a wearing position according to the tilt. The wearing position may refer to a position in which the user wears the wearable device 200. The processor 120 may determine the wearing position of the wearable device 200, based on the wearing hand of the wearable device 200 and the tilt of the wearable device 200. The processor 120 may, in case that the wearing hand of the wearable device 200 is the left hand, determine the wearing position according to the left-hand tilt detection algorithm. The processor 120 may, in case that the wearing hand of the wearable device 200 is the right hand, determine the wearing position according to the right-hand tilt detection algorithm.

In operation 409, the processor 120 may provide a user interfaces corresponding to the wearing position. The user interface may include at least one of text, an image, audio, or a video. The processor 120 may display the user interface through the display 220. Alternatively, the processor 120 may output a voice related to the user interface through the audio module 205 or 208. The processor 120 may measure oxygen saturation while performing operation 405 to operation 409.

According to an embodiment, the processor 120 may, in case that the wearing position corresponds to a first position, provide a first user interface related to the first position. The first position refers to a wearing position of the wearable device 200 that is above the height of the user's heart and is less affected by venous blood and may correspond to a wearing guide position. The first position may correspond to a case where the angle (e.g., θ) between the wrist direction of the wearable device 200 and the ground has a value exceeding a first reference value (e.g., when the wearable device 200 is worn on the left hand, θ>60°) or less than a second reference value (e.g., (e.g., when the wearable device 200 is worn on the right hand, θ<−60°). The first user interface may indicate that oxygen saturation is being measured.

Alternatively, the processor 120 may, in case that the wearing position corresponds to a second position, provide a second user interface related to the second position. The second position may not correspond to the wearing guide position and refer to as a position in which the accuracy of the measurement of oxygen saturation may vary depending on wearing states of the wearable device 200. The second position may correspond to a case where the angle (e.g., θ) between the wrist direction of the wearable device 200 and the ground has a value falling within a reference range (e.g., a value between the first reference value and the second reference value, −60°≤θ≤60°). The second user interface may include a guide indicating that the accuracy of measurement of oxygen saturation is reduced and prompting correct wearing of wearable device 200.

Alternatively, the processor 120 may, in case that the wearing position corresponds to a third position, provide a third user interface related to the third position. The third position may not correspond to the wearing guide position and correspond to a position that may cause problems with oxygen saturation measurement (e.g., an inaccurate value of oxygen saturation). The third position may correspond to a case where the angle (e.g., θ) between the wrist direction of the wearable device 200 and the ground has a value opposite to the first position (e.g., when the wearable device 200 is worn on the left hand, θ<−60°, and when the wearable device 200 is worn on the right hand, θ>60°). The third user interface may include a guide indicating that the measurement of oxygen saturation is impossible and prompting correct wearing of wearable device 200.

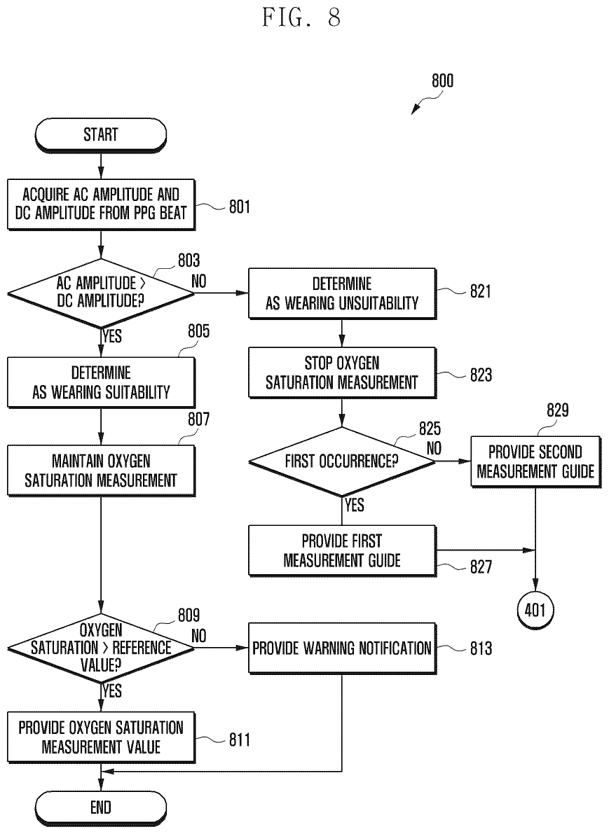

Accordingly, the processor 120 may selectively maintain the oxygen saturation measurement only in case that the wearing position of the wearable device 200 corresponds to the first position or the second position. The processor 120 may, in case that the wearing position of the wearable device 200 corresponds to the first position or the second position, perform operations in FIG. 8. Since oxygen saturation is more likely to be mismeasured when wearing position of the wearable device 200 is the third position, the processor 120 may stop measuring oxygen saturation in case that the wearing position of the wearable device 200 is the third position. The processor 120 may, in case that the wearing position of the wearable device 200 corresponds to the third position, not perform operations in FIG. 8. The processor 120 may, in case that the wearing position of the wearable device 200 corresponds to the third position, stop measuring oxygen saturation to prevent degradation of oxygen saturation measurement quality. The processor 120 may prevent a situation that results in low quality oxygen saturation measurement, in advance.

According to an embodiment, the processor 120 may, in case that a motion of the wearable device 200 is detected before measuring oxygen saturation, determine whether the motion detection is a first occurrence. The processor 120 may provide different guides depending on whether it is the first occurrence (or more than a designated number of times (e.g., three times)). Although it is described below as providing different guides depending on whether the motion detection is a first occurrence, it is possible to provide different guides depending on whether the motion detection has occurred more than a designated number of times (e.g., three times). This is merely an implementation issue, and the disclosure is not limited thereto.

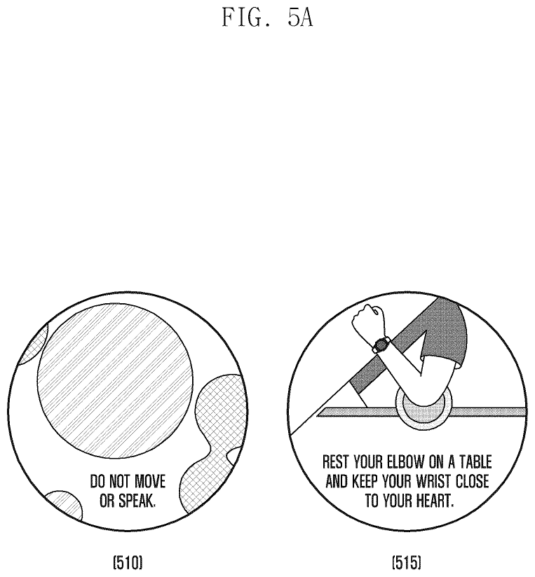

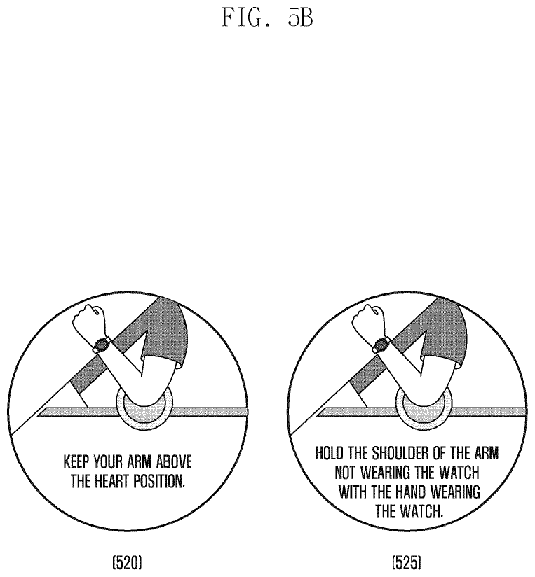

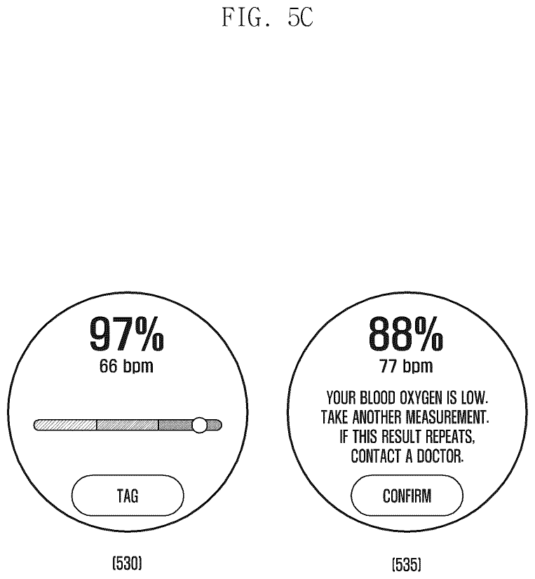

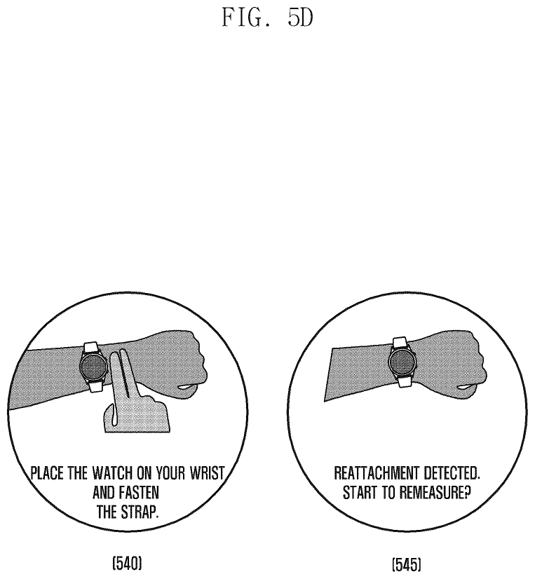

In case that the motion detection is the first occurrence, the processor 120 may provide the first guide. The first guide may include a message, such as “Do not move.” The first guide may include at least one of text, an image, audio, or a video. The processor 120 may, in case that the motion detection is the first occurrence, provide the first user interface 510 of FIG. 5A. The processor 120 may display the first guide through a display (e.g., the display module 160 in FIG. 1 or the display 220 in FIG. 2) of the wearable device 200 or output the first guide using a voice through a speaker (e.g., the sound output module 155 in FIG. 1 or the audio module 205 or 208 in FIG. 2) of the wearable device 200.

In case that the motion detection is not the first occurrence, the processor 120 may provide a second guide. The second guide may be different from the first guide. The second guide may include a message, such as “Take a stable position,” together with a position example. The second guide may include at least one of text, an image, audio, or a video. The processor 120 may, in case that the motion detection is not the first occurrence, provide the second user interface 515 of FIG. 5A. The processor 120 may display the second guide through the display 220 or output the second guide using a voice through the audio module 205 or 208.

FIGS. 5A and 5D are views illustrating an example of a user interface provided in a wearable device according to an embodiment.

FIG. 5A is a view illustrating a user interface provided when a motion of the wearable device is detected.

Referring to FIG. 5A, a processor (e.g., the processor 120 in FIG. 1) of the wearable device (e.g., the electronic device 101 in FIG. 1 or the wearable device 200 in FIG. 2) according to an embodiment may provide a first user interface 510 in case that the motion of the wearable device 200 is the first occurrence. The first user interface 510 may include the first guide such as “Do not move.” The processor 120 may, in case that the motion detection of the wearable device 200 is not the first occurrence, provide a second user interface 515 of FIG. 5A. The second user interface 515 may include a second guide such as “Take a stable position.” or “Rest your elbow on a table and keep your wrist close to your heart.” together with a position example. The first user interface 510 or the second user interface 515 may include at least one of text, an image, audio, or a video.

FIG. 5B is a view illustrating a user interface provided according to a wearing position of the wearable device.

Referring to FIG. 5B, the processor 120 may, in case that the wearing position of the wearable device 200 corresponds to a second position, provide a third user interface 520. The third user interface 520 may include a third guide such as “Keep your arm above the heart position.” The processor 120 may, in case that the wearing position of the wearable device 200 corresponds to the second position or the third position, provide a fourth user interface 525. The fourth user interface 525 may include a message, such as “Hold the shoulder of the arm not wearing the watch (e.g., the wearable device 200) with the hand wearing the watch,” together with a position example. The third user interface 520 or the fourth user interface 525 may include at least one of text, an image, audio, or a video. The example of the third guide or the fourth guide is for illustrative purposes of the disclosure and may include different images or text depending on the wearing position.

FIG. 5C is a view illustrating a user interface related to oxygen saturation measurement in the wearable device.

Referring to FIG. 5C, the processor 120 may provide different user interfaces depending on the measured oxygen saturation. The processor 120 may, in case that the measured oxygen saturation corresponds to a normal value, provide a fifth user interface 530 including the measured oxygen saturation. The fifth user interface 530 may include the measured oxygen saturation and a tag button. The processor 120 may, in case that the measured oxygen saturation does not correspond to a normal value, provide a sixth user interface 535 including the measured oxygen saturation and a warning notification. The sixth user interface 535 may include the measured oxygen saturation and a warning notification (e.g., “Your blood oxygen is low. Take another measurement. If this result repeats, contact a doctor.”). The fifth user interface 530 or the sixth user interface 535 may include at least one of text, an image, audio, or a video.

FIG. 5D is a view illustrating a user interface related to oxygen saturation measurement in the wearable device.

Referring to FIG. 5D, the processor 120 may, in case that the quality of oxygen saturation is low (e.g., wearing unsuitability), provide different user interfaces. The processor 120 may, in case that low oxygen saturation quality is first detected, may provide a seventh user interface 540, such as “Place the watch on your wrist and fasten the strap.” The seventh user interface 540 may include information on a method for correct wearing, together with an example of correct wearing. The processor 120 may, in case that low oxygen saturation quality is not first detected, provide an eighth user interface 545. The eighth user interface 545 may include a guide such as “Remove and reattach the device” or “Reattachment detected. Remeasure oxygen saturation?” or “Reattachment detected. Start to remeasure?”. The seventh user interface 540 or the eighth user interface 545 may include at least one of text, an image, audio, or a video.

The first user interface 510 to the eighth user interface 545 are examples provided to aid in understanding the disclosure and should not be construed as limiting the scope of the disclosure.

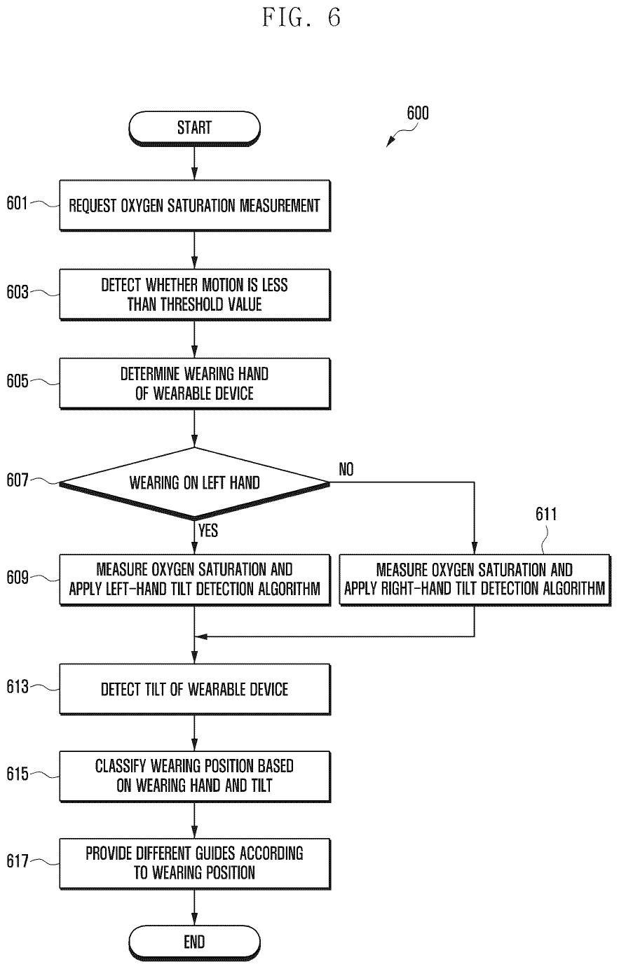

FIG. 6 is a flowchart 600 illustrating a method of classifying wearing positions according to a wearing hand in a wearable device according to an embodiment. Operations in FIG. 6 may embody operations in FIG. 4.

Referring to FIG. 6, in operation 601, a processor (e.g., the processor 120 in FIG. 1) of the wearable device (e.g., the electronic device 101 in FIG. 1 or the wearable device 200 in FIG. 2) according to an embodiment may receive a request for oxygen saturation measurement. The user may wear the wearable device 200 and then request the oxygen saturation measurement. For example, the processor 120 may receive, from the user, receive selecting (e.g., touching) an oxygen saturation measurement button from a measurement menu or a voice input such as “Measure oxygen saturation.”

In operation 603, the processor 120 may detect whether a motion is less than or equal to a threshold value. The processor 120 may detect whether there is no motion of the wearable device 200 by using a sensor module (e.g., the sensor module 176 in FIG. 1) included in the wearable device 200. For example, the sensor module 176 may correspond to an inertial sensor, and the inertial sensor may include an acceleration sensor and a gyro sensor. According to one or more embodiments, the inertial sensor is always operated and thus the processor 120 may acquire motion data from the inertial sensor in real time. The processor 120 may detect whether there is a motion of the wearable device 200, based on the motion data. Detecting whether a motion is less than or equal to a threshold value may correspond to identifying whether there is no motion. The processor 120 may, in case that the motion is less than or equal to the threshold value, perform operation 605.

In operation 605, the processor 120 may determine a wearing hand of the wearable device 200. The processor 120 may, in case that the motion of the wearable device 200 has not been detected, detect a wearing hand of the wearable device 200, based on the motion data acquired during a process in which the user raises a hand for measuring oxygen saturation. Alternatively, the processor 120 may provide a user interface for querying the user whether the wearable device 200 is worn on the left or right hand and acquire information about the wearing hand from the user. Alternatively, the processor 120 may acquire information about the wearing hand of the wearable device 200 from configuration information of the wearable device 200. The configuration information may be acquired by directly inputting information about the wearing hand of the wearable device 200.

In operation 607, the processor 120 may determine whether the wearing hand of the wearable device 200 is the left hand. The drawing shows the determination of whether the wearing hand is the left hand, but it is also possible to determine whether the wearing hand is the right hand. In general, since the user may be more likely to wear the wearable device on the left hand, it may determine whether the wearing hand is the left hand. The processor 120 may perform operation 609 in case that the wearing hand of the wearable device 200 is the left hand, and perform operation 611 in case that the wearing hand of the wearable device 200 is the right hand.

In case that the wearing hand of the wearable device 200 is the left hand, in operation 609, the processor 120 may measure oxygen saturation and apply the left-hand tilt detection algorithm. This may be because the direction of the wearable device 200 alone does not indicate whether a current position of the wearable device 200 is higher or lower than the user's heart. For example, this may be to distinguish when the user is wearing the wearable device 200 on the left wrist and is raising the left hand, and when the user is wearing the wearable device 200 on the right wrist and is lowering the right wrist. For example, in case that the wearing hand of the wearable device 200 is the left hand, the angle (e.g., θ) between the wrist direction of the wearable device 200 and the ground may correspond to a determined positive value (e.g., θ>60°, exceeding a first reference value). Accordingly, the processor 120 may apply a different tilt detection algorithm depending on the wearing hand of the wearable device 200.

According to an embodiment, the processor 120 may start oxygen saturation measurement by means of the PPG sensor (e.g., the sensor module 176 in FIG. 1). The sensor module 176 may further include a PPG sensor. The processor 120 may acquire a PPG signal from the PPG sensor and calculate oxygen saturation by using a ratio of AC to DC components for each wavelength included in the acquired PPG signal. The method for calculating oxygen saturation is well known in the art and will not be described in detail.

In case that the wearing hand of the wearable device 200 is the right hand, in operation 611, the processor 120 may measure oxygen saturation and apply the right-hand tilt detection algorithm. In case that the wearing hand of the wearable device 200 is the right hand, the angle (e.g., θ) between the wrist direction of the wearable device 200 and the ground may correspond to a determined negative value (e.g., θ<−60°, less than a second reference value). The processor 120 may perform operation 611 and then perform operation 613.

In operation 613, the processor 120 may a tilt of the wearable device 200. The processor 120 may, in case that the wearing hand of the wearable device 200 is the left hand, apply the left-hand tilt detection algorithm to detect the tilt of the wearable device 200. The processor 120 may, in case that the wearing hand of the wearable device 200 is the right hand, apply the right-hand tilt detection algorithm to detect the tilt of the wearable device 200.

In operation 615, the processor 120 may determine a wearing position according to the wearing hand and the tilt. The wearing position may refer to a position in which the user wears the wearable device 200. The processor 120 may determine the wearing position of the wearable device 200, based on the wearing hand of the wearable device 200 and the tilt of the wearable device 200. The processor 120 may, in case that the wearing hand of the wearable device 200 is the left hand, determine the wearing position according to the left-hand tilt detection algorithm. The processor 120 may, in case that the wearing hand of the wearable device 200 is the right hand, determine the wearing position according to the right-hand tilt detection algorithm.

In operation 615, the processor 120 may provide a different wearing guide depending on the wearing position of the wearable device 200. The wearing guide may include at least one of text, an image, audio, or a video. The processor 120 may display the wearing guide through the display 220 or output a voice related to the wearing guide through the audio module 205 or 208. The processor 120 may measure oxygen saturation while performing operation 609 to operation 617.

According to an embodiment, the processor 120 may, in case that the wearing position corresponds to the first position, provide a first wearing guide related to the first position. The first position refers to a wearing position of the wearable device 200 that is above the height of the user's heart and is less affected by venous blood and may correspond to a wearing guide position. The first position may correspond to a case where the angle (e.g., θ) between the wrist direction of the wearable device 200 and the ground has a value exceeding a first reference value (e.g., when the wearable device 200 is worn on the left hand, θ>60°) or less than a second reference value (e.g., when the wearable device 200 is worn on the right hand, θ>60°). The first wearing guide may indicate that oxygen saturation is being measured. Alternatively, the processor 120 may, in case that the wearing position corresponds to the second position, provide a second wearing guide related to the second position.

The second position may not correspond to the wearing guide position and refer to as a position in which the accuracy of the measurement of oxygen saturation may vary depending on wearing states of the wearable device 200. The second position may correspond to a case where the angle (e.g., θ) between the wrist direction of the wearable device 200 and the ground has a value falling within a reference range (e.g., a value between the first reference value and the second reference value, −60≤θ≤60°). The second wearing guide may include a guide indicating that the accuracy of measurement of oxygen saturation is reduced, and prompting correct wearing of wearable device 200. The processor 120 may, in case that the wearing position of the wearable device 200 corresponds to the second position, provide the third user interface 520 or the fourth user interface 525 in FIG. 5B.

Alternatively, the processor 120 may, in case that the wearing position corresponds to the third position, provide a third wearing guide related to the third position. The third position may not correspond to the wearing guide position and correspond to a position that may cause problems with oxygen saturation measurement (e.g., an inaccurate value of oxygen saturation). The third position may correspond to a case where the angle (e.g., θ) between the wrist direction of the wearable device 200 and the ground has a value opposite to the first position (e.g., when the wearable device 200 is worn on the left hand, θ<60°, and when the wearable device 200 is worn on the right hand, θ>60°). The third wearing guide may include a guide indicating that the measurement of oxygen saturation is impossible, and prompting correct wearing of wearable device 200. The processor 120 may, in case that the wearing position of the wearable device 200 corresponds to the second position, provide the fourth user interface 525 in FIG. 5B.

Accordingly, the processor 120 may selectively maintain the oxygen saturation measurement only in case that the wearing position of the wearable device 200 corresponds to the first position or the second position. The processor 120 may, in case that the wearing position of the wearable device 200 corresponds to the first position or the second position, perform operations in FIG. 8. Since oxygen saturation is more likely to be mismeasured when wearing position of the wearable device 200 is the third position, the processor 120 may stop measuring oxygen saturation in case that the wearing position of the wearable device 200 is the third position. The processor 120 may, in case that the wearing position of the wearable device 200 corresponds to the third position, not perform operations in FIG. 8. The processor 120 may, in case that the wearing position of the wearable device 200 corresponds to the third position, stop measuring oxygen saturation to prevent degradation of oxygen saturation measurement quality. The processor 120 may prevent a situation that results in low quality oxygen saturation measurement, in advance.

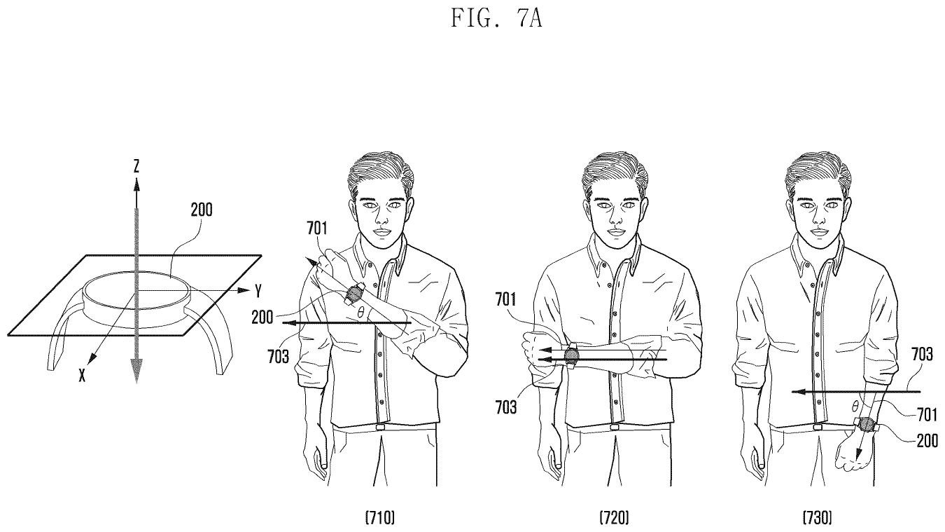

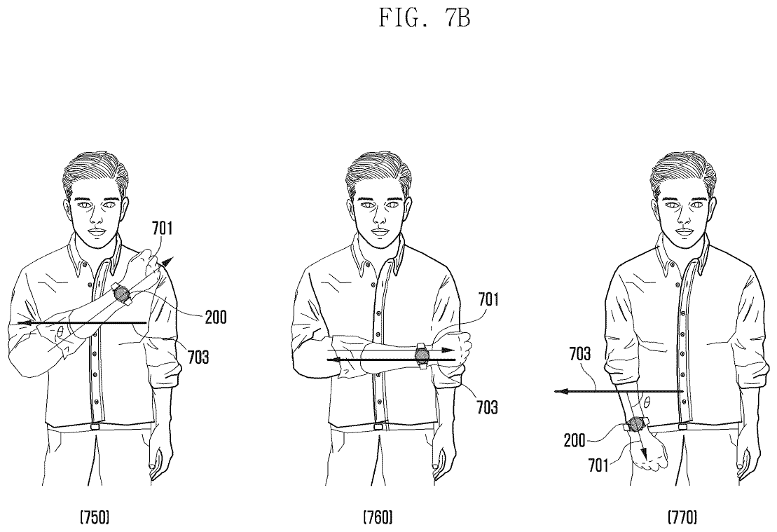

FIGS. 7A and 7B are views illustrating an example of classifying wearing positions according to a wearing hand in a wearable device according to an embodiment.

FIG. 7A is a view illustrating an example of classifying wearing positions in case that the wearable device is worn on the left hand.

Referring to FIG. 7A, the processor (e.g., the processor 120 in FIG. 1) of the wearable device (e.g., the electronic device 101 in FIG. 1 or the wearable device 200 in FIG. 2) according to an embodiment may, in case that the wearing hand of the wearable device 200 is the left hand, classify wearing positions into at least one of a first wearing position 710, a second wearing position 720, or a third wearing position 730 according to the tilt of the wearable device 200. The processor 120 may convert motion data (e.g., x, y, or z) acquired from an inertial sensor (e.g., the sensor module 176 in FIG. 1) into an Euler angle (e.g., roll, pitch, or yaw), and detect the tilt of the wearable device 200 based on the converted Euler angle.

The first wearing position 710 refers to a wearing position of the wearable device 200 that is above the height of the user's heart and is less affected by venous blood and may correspond to a wearing guide position. In the first wearing position 710, the angle (e.g., θ) between the wrist direction 701 of the wearable device 200 and the ground 703 may exceed a determined positive value (e.g., θ>60°).

The second wearing position 720 may not correspond to the wearing guide position and refer to as a position in which the accuracy of the measurement of oxygen saturation may vary depending on wearing states of the wearable device 200. In the second wearing position 720, the angle (e.g., θ) between the wrist direction 701 of the wearable device 200 and the ground 703 may have a value within a determined value range (−60≤θ≤60°). In the second wearing position 720, the wrist direction of the wearable device 200 and the ground direction may be parallel (e.g., θ=0). The processor 120 may provide a guide prompting the user to correctly wear the wearable device 200 since the accuracy of oxygen saturation measurement is reduced in case that the wearing position of the wearable device 200 corresponds to the second wearing position 720.

The processor 120 may, in case that the wearing position of the wearable device 200 corresponds to the first wearing position 710 or the second wearing position 720, perform operations in FIG. 8.

The third wearing position 730 may not correspond to the wearing guide position and correspond to a position that may cause problems with oxygen saturation measurement (e.g., an inaccurate value of oxygen saturation). In the third wearing position 730, the angle (e.g., θ) between the wrist direction 701 of the wearable device 200 and the ground 703 may have a determined negative value (e.g., θ<−60°). Since oxygen saturation is more likely to be mismeasured in the third wearing position 730, the processor 120 may stop measuring oxygen saturation in case that the wearing position of the wearable device 200 corresponds to the third wearing position 730. The processor 120 may, in case that the wearing position of the wearable device 200 corresponds to the third wearing position 730, not perform operations in FIG. 8. The processor 120 may, in case that the wearing position of the wearable device 200 corresponds to the third wearing position 730, stop measuring oxygen saturation to prevent degradation of oxygen saturation measurement quality. The processor 120 may prevent a situation that results in low quality oxygen saturation measurement, in advance.

FIG. 7B is a view illustrating an example of classifying wearing positions in case that the wearable device is worn on the right hand.

Referring to FIG. 7B, the processor 120 may, in case that the wearing hand of the wearable device 200 is the right hand, classify wearing positions into at least one of a fourth wearing position 750, a fifth wearing position 760, or a sixth wearing position 770 according to the tilt of the wearable device 200. The fourth wearing position 750 refers to a wearing position of the wearable device 200 that is above the height of the user's heart and is less affected by venous blood and may correspond to a wearing guide position. In the fourth wearing position 750, the angle (e.g., θ) between the wrist direction of the wearable device 200 and the ground may have a negative value (e.g., θ>−60°).

The fifth wearing position 760 may not correspond to the wearing guide position and refer to as a position in which the accuracy of the measurement of oxygen saturation may vary depending on wearing states of the wearable device 200. In the fifth wearing position 760, the angle (e.g., θ) between the wrist direction 701 of the wearable device 200 and the ground 703 may have a value between a specific positive value and a specific negative value (−60°≤θ≤60°). In the fifth wearing position 760, the wrist direction of the wearable device 200 and the ground direction may be parallel (e.g., θ=0). The processor 120 may provide a guide prompting the user to correctly wear the wearable device 200 since the accuracy of oxygen saturation measurement is reduced in case that the wearing position of the wearable device 200 corresponds to the fifth wearing position 760.

The processor 120 may, in case that the wearing position of the wearable device 200 corresponds to the fourth wearing position 750 or the fifth wearing position 760, perform operations in FIG. 8.

The sixth wearing position 770 may not correspond to the wearing guide position and correspond to a position that may cause problems with oxygen saturation measurement (e.g., an inaccurate value of oxygen saturation). In the sixth wearing position 770, the angle (e.g., θ) between the wrist direction 701 of the wearable device 200 and the ground 703 may have a positive value (e.g., θ>60°). Since oxygen saturation is more likely to be mismeasured in the sixth wearing position 770, the processor 120 may stop measuring oxygen saturation in case that the wearing position of the wearable device 200 corresponds to the sixth wearing position 770. The processor 120 may, in case that the wearing position of the wearable device 200 corresponds to the sixth wearing position 770, not perform operations in FIG. 8. The processor 120 may, in case that the wearing position of the wearable device 200 corresponds to the sixth wearing position 770, stop measuring oxygen saturation to prevent degradation of oxygen saturation measurement quality. The processor 120 may prevent a situation that results in low quality oxygen saturation measurement, in advance.

FIG. 8 is a flowchart 800 illustrating a method of providing a user interface depending on wearing suitability in a wearable device according to an embodiment. FIG. 8 may show operations performed after operation 409 in FIG. 4 or operation 617 in FIG. 6.

Referring to FIG. 8, in operation 801, a processor (e.g., the processor 120 in FIG. 1) of the wearable device (e.g., the electronic device 101 in FIG. 1 or the wearable device 200 in FIG. 2) according to an embodiment may acquire an amplitude of AC and an amplitude of DC from a PPG beat (or PPG signal). The processor 120 may measure oxygen saturation by means of the PPG sensor (e.g., the sensor module 176 in FIG. 1). The processor 120 may acquire the PPG signal from the PPG sensor and acquire the amplitude of AC and the amplitude of DC from the acquired PPG signal. The PPG signal may be modulated by respiration and thus the processor 120 may determine the wearing suitability of the wearable device 200 by determining whether the modulation caused by respiration is significant. The processor 120 may acquire the amplitude of AC and the amplitude of DC from the acquired PPG signal to determine whether the PPG signal include demodulation caused by respiration.

According to an embodiment, the processor 120 may use a morphological similarity (Pearson correlation coefficient) between PPG signals. For example, the processor 120 may determine a morphological similarity within an identical wavelength, or determine a morphological similarity between wavelengths. The processor 120 may, in case that the morphological similarity exceeds a determined reference value, perform operation 805. Alternatively, the processor 120 may compare a waveform template stored in a memory (e.g., the memory 130 in FIG. 1) and the measured PPG signal to determine a similarity between the waveform template and the measured PPG signal. The waveform template may correspond to an ideal waveform acquired when measuring oxygen saturation using the correct wearing method. The processor 120 may, in case that a similarity between two signals exceeds a determined reference value, perform operation 805.

In operation 803, the processor 120 may determine whether the amplitude of AC exceeds the amplitude of DC. The AC amplitude may be defined as the width of the PPG signal between systole and diastole in a single beat, and the DC amplitude may be defined as the width of the PPG signal between diastole or systole over multiple beats. A length of a window for determining the DC amplitude may be determined to include at least one cycle of modulation by respiration. For example, in case that the wearable device 200 is worn such that the user's body (e.g., wrist) applies moderate pressure to the wearable device 200, the PPG signal may be acquired primarily as an arterial blood waveform. In this case, in case that the amplitudes of AC and DC are acquired for a determined period (e.g., 6) of a PPG signal period, the AC amplitude may be greater than the DC amplitude (e.g., a ratio of AC amplitude/DC amplitude is high) because a change amount in DC is not large. The determined period may be longer than a respiration cycle. However, in case that the wearable device 200 is not worn such that the user's body (e.g., wrist) applies moderate pressure to the wearable device 200 (e.g., in case of loosely wearing), a venous blood waveform may be introduced into the PPG signal by respiration. In this case, in case that the amplitudes of AC and DC are acquired for a determined period of the PPG signal period, the AC amplitude may be less than the DC amplitude (e.g., a ratio of AC amplitude/DC amplitude is low) because a change amount in DC is large (e.g., a multiple of AC).

The processor 120 may, in case that the AC amplitude exceeds the DC amplitude, perform operation 805, and in case that the AC amplitude is less than or equal to the DC amplitude, perform operation 821.

In case that the AC amplitude exceeds the DC amplitude, in operation 805, the processor 120 may determine it corresponds to wearing suitability. The wearing suitability may refer to a state where the wearable device 200 is worn such that the user's body applies moderate pressure to the wearable device 200. That is, the wearing suitability may refer to a state where the user is correctly wearing the wearable device 200 when oxygen saturation is measured. The wearing suitability may refer to a state where a degree of contact between the user's body and the wearable device 200 is strong, such that there is little (or no) space (or gap) between the user's body and the wearable device 200.

In operation 807, the processor 120 may maintain oxygen saturation measurement. The processor 120 may, in case that the wearing state of the wearable device 200 corresponds to the wearing suitability, continuously measure oxygen saturation.

In operation 809, the processor 120 may determine whether the oxygen saturation exceeds a reference value. The oxygen saturation may be used to measure an amount of oxygen in the blood so as to indirectly determine whether oxygen is being adequately delivered to the respiratory system of the body. For example, an oxygen saturation level of 95% or higher is considered normal in cases without respiratory problems. Accordingly, the reference value may be configured to a value (or range of values, (e.g., 90% to 95%)) that is determined to be normal. Alternatively, the reference value may be configured to be changed by the user. For example, in case that the user has a respiratory disorder, the reference value may be configured slightly lower. In case that the user manually configures the reference value, the reference value may be stored in a memory (e.g., the memory 130 of FIG. 1) of the wearable device 200. However, a lower bound on the reference value (e.g., 80% or more) may be established to prevent excessive mismeasurement.

The processor 120 may, in case that the oxygen saturation exceeds the reference value, perform operation 811, and in case that the oxygen saturation is equal to or less than the reference value, perform operation 813.

In case that the oxygen saturation exceeds the reference value (e.g., the quality of the oxygen saturation is high), in operation 811, the processor 120 may provide an oxygen saturation measurement value. The processor 120 may display the oxygen saturation value (or figure) through a display (e.g., the display module 160 in FIG. 1, or the display 220 in FIG. 2). Alternatively, the processor 120 may output the oxygen saturation value as a voice through a speaker (e.g., the sound output module 155 in FIG. 1 or the audio module 205 or 208 in FIG. 2) of the wearable device 200. The processor 120 may, in case that the oxygen saturation exceeds the reference value, provide the fifth user interface 530 of FIG. 5C.

In case that the oxygen saturation is less than or equal to the reference value (e.g., the quality of oxygen saturation is low), in operation 811, the processor 120 may provide a warning notification. The processor 120 may display the oxygen saturation measurement value (or figure) and the warning notification (e.g., it may be inaccurate if the wearing hand is down toward the ground) through the display 220, or may output same as a voice through the audio modules 205 or 208. The processor 120 may, in case that the oxygen saturation is less than or equal to the reference value, provide the sixth user interface 535 of FIG. 5C.

In case that the AC amplitude is less than or equal to the DC amplitude, in operation 821, the processor 120 may determine it corresponds to wearing unsuitability. The wearing unsuitability may refer to a state where the wearable device 200 is not worn such that the user's body applies moderate pressure to the wearable device 200. That is, the wearing unsuitability may refer to a state where the user is not correctly wearing the wearable device 200 when oxygen saturation is measured. For example, the wearing unsuitability may refer to a state where a degree of contact between the user's body and the wearable device 200 is weak, such that there is wide space between the user's body and the wearable device 200.

According to an embodiment, the processor 120 may use an algorithm without an oxygen saturation output value to determine a signal quality of the oxygen saturation through a coverage within a measurement window section. For example, in case that the oxygen saturation value is output at a frequency of less than 10% within a configured period (e.g., 5 seconds), the processor 120 may determine that it corresponds to wearing unsuitability. In case that the oxygen saturation output value does not exist, the processor 120 may determine the signal quality of the oxygen saturation based on whether the coverage is high or low.

In operation 823, the processor 120 may stop oxygen saturation measurement. The processor 120 may, in case that the wearing state of the wearable device 200 corresponds to the wearing unsuitability, stop the oxygen saturation measurement. Because the measured oxygen saturation includes a venous blood waveform due to a wearing defect of the wearable device 200, the processor 120 may determine that the signal quality of the oxygen saturation is low (e.g., difficulty providing an accurate oxygen saturation value) and no longer measure the oxygen saturation.

In operation 825, the processor 120 may determine whether interruption in the oxygen saturation measurement is a first-time occurrence. Although it is described below as providing different measurement guides depending on whether the interruption in oxygen saturation measurement is a first occurrence, it is possible to provide different measurement guides depending on whether the interruption in oxygen saturation measurement has occurred more than or equal to a designated number of times (e.g., three times). This is merely one possible implementation, and the present disclosure is not limited thereto.

In case that the interruption in oxygen saturation measurement is the first occurrence, in operation 827, the processor 120 may provide a first measurement guide. The first measurement guide may include a message, such as “Tighten the watch strap” or “Move the wearable device up toward the body (e.g., below or above the elbow).” The first measurement guide may include at least one of text, an image, audio, or a video. The processor 120 may, in case that the interruption in oxygen saturation measurement is the first occurrence, provide the seventh user interface 540 of FIG. 5D. The processor 120 may display the first measurement guide through the display 220 or output the first measurement guide using a voice through the audio module 205 or 208. The processor 120 may perform operation 827 and then return to operation 401.

In case that the interruption in oxygen saturation measurement is not the first occurrence (e.g., multiple occurrences), in operation 829, the processor 120 may provide the second measurement guide. The second measurement guide may be different from the first measurement guide. The second measurement guide may include a message, such as “Remove and reattach the wearable device” or “Switch the wearing hand (e.g., guide wearing on the opposite hand).” The second measurement guide may include at least one of text, an image, audio, or a video. The processor 120 may, in case that the interruption in oxygen saturation measurement is not the first occurrence, provide the eighth user interface 545 of FIG. 5D. The processor 120 may display the second measurement guide through the display 220 or output the second measurement guide using a voice through the audio module 205 or 208. The processor 120 may perform operation 829 and then return to operation 401.

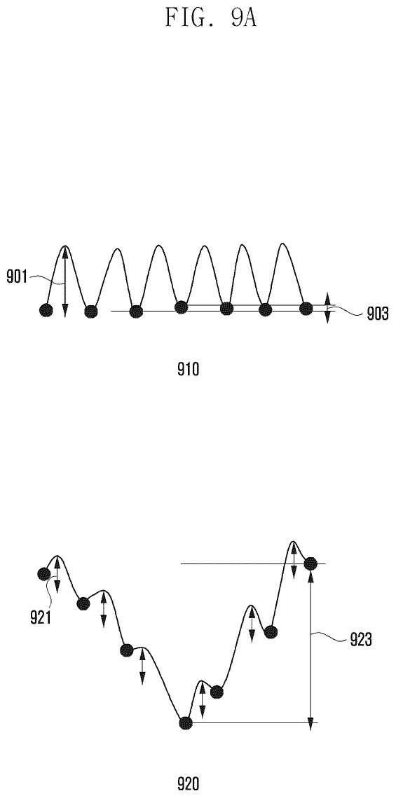

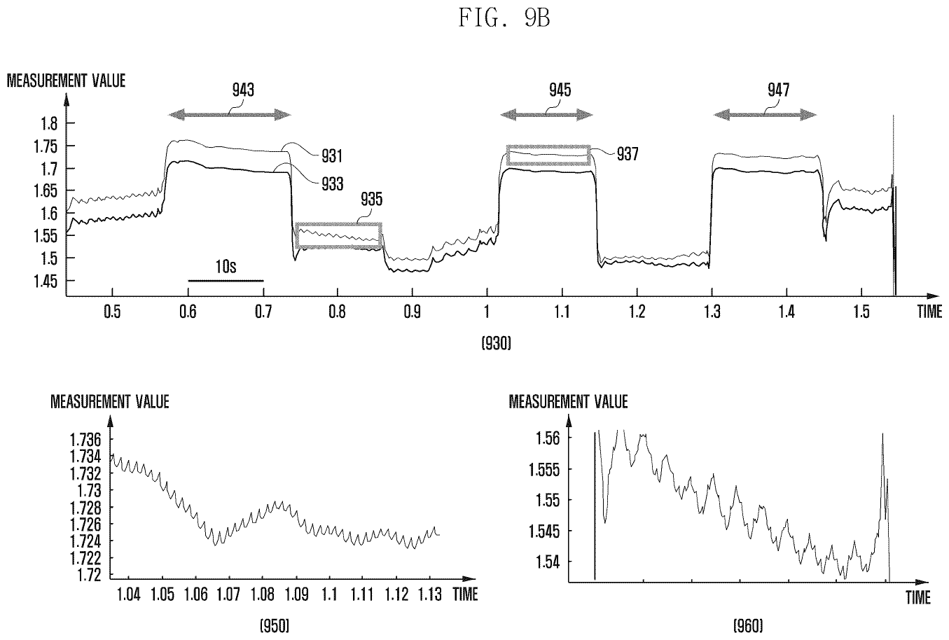

FIGS. 9A and 9B are views illustrating an example of classifying wearing suitability of a wearable device according to an embodiment.

FIG. 9A is a view illustrating an example of a PPG signal acquired according to a wearing state of the wearable device.

Referring to FIG. 9A, a processor (e.g., the processor 120 in FIG. 1) of the wearable device (e.g., the electronic device 101 in FIG. 1 or the wearable device 200 in FIG. 2) according to an embodiment may acquire an amplitude of AC and an amplitude of DC from a PPG beat (or PPG signal). The AC amplitude may be defined as the width of the PPG signal between systole and diastole in a single beat, and the DC amplitude may be defined as the width of the PPG signal between diastole or systole over multiple beats. A length of a window for determining the DC amplitude may be determined to include at least one cycle of modulation by respiration. For example, in case that the user is wearing the wearable device 200 correctly (e.g., wearing suitability) when measuring oxygen saturation, the processor 120 may acquire a first PPG signal 910. In the first PPG signal 910, the AC amplitude 901 may be greater than the DC amplitude 903 (e.g., a ratio of AC amplitude/DC amplitude is high). However, in case that the user is wearing the wearable device 200 incorrectly (e.g., wearing unsuitability) when measuring oxygen saturation, the processor 120 may acquire a second PPG signal 920. In the second PPG signal 920, the AC amplitude 921 may be less than the DC amplitude 923 (e.g., a ratio of AC amplitude/DC amplitude is low).

FIG. 9B is a graph illustrating an example of a PPG signal acquired according to a wearing state of the wearable device.

Referring to FIG. 9B, a first signal graph 930 depicts a PPG signal acquired according to the wearing state of the wearable device 200. The first PPG signal 931 may be acquired when infrared is used for the light-emitting unit, and the second PPG signal 933 may be acquired when RED is used for the light-emitting unit. The first PPG signal 931 and the second PPG signal 933 may show similar patterns according to the wearing state of the wearable device 200. For example, in case that the user is wearing the wearable device 200 correctly, PPG signals, such as a first signal period 943, a second signal period 945, and a third signal period 947, may be acquired. In the first signal period 943, the second signal period 945, and the third signal period 947, the AC amplitude may be greater than the DC amplitude (e.g., the ratio of AC amplitude/DC amplitude is high). In case that the user is not wearing the wearable device 200 correctly, a PPG signal, such as a fourth signal period 935, may be acquired. In the fourth signal period 935, the AC amplitude may be less than the DC amplitude (e.g., the ratio of AC amplitude/DC amplitude is low).

In addition, a second graph 950 may depict a PPG signal acquired when the user is wearing the wearable device 200 correctly. In case that the amplitudes of AC and DC are acquired from the PPG signal as shown in the second graph 950, the AC amplitude may be greater than the DC because a change amount in DC is not large. A third graph 960 may depict a PPG signal acquired when the user is not wearing the wearable device 200 correctly. In case that the amplitudes of AC and DC are acquired from the PPG signal as shown in the third graph 960, the AC amplitude may be less than the DC amplitude because a change amount in DC is large.

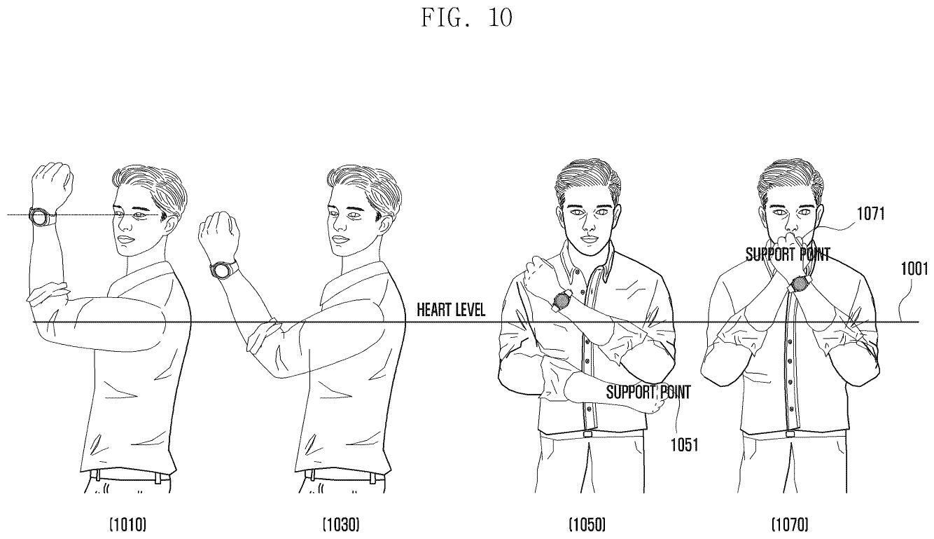

FIG. 10 is a view illustrating an example of providing a guide associated with oxygen saturation measurement in a wearable device according to an embodiment.

Referring to FIG. 10, a processor (e.g., the processor 120 in FIG. 1) of the wearable device (e.g., the electronic device 101 in FIG. 1 or the wearable device 200 in FIG. 2) according to an embodiment may, in case that a quality of a signal is not good, additionally provide at least one of a first measurement guide 1010 to a fourth measurement guide 1070. For example, for higher signal quality, the processor 120 may provide the first measurement guide 1010 to guide the user to place the wearable device 200 up to the eye level. The processor 120 may provide the second measurement guide 1030 to guide the user to place the wearable device 200 above the heart level 1001 to reduce an effect of venous blood. For higher signal quality, the processor 120 may provide the third measurement guide 1050 or the fourth measurement guide 1070 to guide the user to support the wearable device 200 using the opposite hand (e.g., a hand that is not wearing the wearable device 200) to avoid shaking. The third measurement guide 1050 may show a support point (e.g., an elbow 1051) to minimize shaking of the wearable device 200 using the opposite hand. The fourth measurement guide 1070 may show a support point (e.g., the back of the other hand 1057) to minimize shaking of the wearable device 200 using the opposite hand.

According to an embodiment, an operation method of a wearable device 200 may include an operation of, in response to a request for measurement of oxygen saturation, acquiring motion data from an inertial sensor (e.g., sensor module 176) included in the wearable device, an operation of, in case that it is determined based on the motion data that there is no motion in a wearable device, determining a wearing hand on which the wearable device is worn, based on the motion data, an operation of detecting a tilt of the wearable device while measuring the oxygen saturation by means of a PPG sensor (e.g., sensor module 176) included in the wearable device, an operation of classifying wearing positions of the wearable device based on the wearing hand and the tilt, and an operation of providing different user interfaces according to the wearing positions.

The method may further include an operation of, in case that the wearing hand corresponds to the left hand, applying a left hand tilt detection algorithm, an operation of determining whether an angle between a wrist direction of the wearable device and the ground exceeds a first reference value according to the left hand tilt detection algorithm, and an operation of classifying the wearing position of the wearable device according to a result of the determination.

The method may further include an operation of, in case that the wearing hand corresponds to the right hand, applying a right hand tilt detection algorithm, an operation of determining whether the angle between the wrist direction of the wearable device and the ground is less than a second value according to the right hand tilt detection algorithm, and an operation of classifying the wearing position of the wearable device according to a result of the determination.

The method may perform one of an operation of, in case that the wearing position corresponds to a first position, providing a first user interface associated with the first position, an operation of, in case that the wearing position corresponds to a second position, providing a second user interface associated with the second position, and an operation of, in case that the wearing position corresponds to a third position, providing a third user interface associated with the third position, wherein the first position corresponds to a case where the angle between the wrist direction of the wearable device and the ground having a value exceeding a first reference value or less than a second reference value, the second position corresponds to a case where the angle between the wrist direction of the wearable device and the ground has a value between the first reference value and the second reference value, and the third position corresponds to a case where the angle between the wrist direction of the wearable device and the ground has a value opposite to the first position.

The method may further include an operation of, in case that the wearing position corresponds to the first position or the second position, maintaining oxygen saturation measurement and an operation of, in case that the wearing position corresponds to the third position, stopping oxygen saturation measurement.

The method may include an operation of acquiring an amplitude of AC and an amplitude of DC from a PPG signal acquired from the PPG sensor, an operation of, in case that the amplitude of the AC exceeds the amplitude of the DC, determining that a wearing state of the wearable device is suitable, and an operation of, in case that the amplitude of the AC is equal to or less than the amplitude of the DC, determining that the wearing state of the wearable device is unsuitable.

The method may include an operation of, when it is determined that the wearing state of the wearable device is suitable, maintaining the oxygen saturation measurement, an operation of determining whether the measured oxygen saturation exceeds a reference value, an operation of, in case that the measured oxygen saturation exceeds the reference value, providing an oxygen saturation measurement value, and in case that the measured oxygen saturation is equal to or less than the reference value, providing the oxygen saturation measurement value and a warning notification, an operation of, when it is determined that the wearing state of the wearable device is unsuitable, stopping the oxygen saturation measurement, and an operation of, in case that the number of oxygen saturation measurement stops corresponds to a designated number of times, providing a first measurement guide, and in case that the number of oxygen saturation measurement stops does not correspond to the designated number of times, providing a second measurement guide.

The embodiments disclosed in the specification and the drawings are merely presented as specific examples to easily explain the technical features and help understanding of the disclosure and are not intended to limit the scope of the disclosure. Therefore, the scope of the disclosure should be construed as encompassing all changes or modifications derived from the technical ideas of the disclosure in addition to the embodiments disclosed herein.

What is claimed is:

1. A wearable device comprising:
an inertial sensor;
a photoplethysmography (PPG) sensor;
a display module;
a processor operatively coupled to at least one of the inertial sensor, the PPG sensor, and the display module; and
a memory connected electrically to the processor and configured to store instructions executable by the processor, wherein the instructions, when executed by the processor, cause the wearable device to:
in response to a request for measurement of oxygen saturation, acquire motion data from the inertial sensor;
responsive to a determination, based on the motion data, that there is no motion of the wearable device, determine a wearing hand on which the wearable device is worn, based on the motion data;
detect a tilt of the wearable device during the measurement of oxygen saturation by means of the PPG sensor;
classify wearing positions of the wearable device, based on the wearing hand and the tilt; and
provide different user interfaces according to the wearing positions.

2. The wearable device of claim 1, wherein the instructions are configured to, when executed by the processor, cause the wearable device to:
in case that the wearing hand corresponds to a left hand, apply a left-hand tilt detection algorithm; and
in case that the wearing hand corresponds to a right hand, apply a right-hand tilt detection algorithm.

3. The wearable device of claim 2, wherein the instructions are configured to, when executed by the processor, cause the wearable device to:
determine whether an angle between a wrist direction of the wearable device and a ground exceeds a first reference value according to the left-hand tilt detection algorithm; and
classify the wearing positions of the wearable device according to a result of the determination.

4. The wearable device of claim 2, wherein the instructions are configured to, when executed by the processor, cause the wearable device to:
determine whether an angle between a wrist direction of the wearable device and a ground is less than a second reference value according to the right-hand tilt detection algorithm; and
classify the wearing positions of the wearable device according to a result of the determination.

5. The wearable device of claim 1, wherein the instructions are configured to, when executed by the processor, cause the wearable device to:
in case that a wearing position corresponds to a first position, provide a first user interface associated with the first position;
in case that the wearing position corresponds to a second position, provide a second user interface associated with the second position; and
in case that the wearing position corresponds to a third position, provide a third user interface associated with the third position,
wherein the first position corresponds to a case where an angle between a wrist direction of the wearable device and a ground has a first value exceeding a first reference value or less than a second reference value,
wherein the second position corresponds to a case where the angle between the wrist direction of the wearable device and the ground has a second value between the first reference value and the second reference value, and
wherein the third position corresponds to a case where the angle between the wrist direction of the wearable device and the ground has a third value opposite to the first position.

6. The wearable device of claim 5, wherein the instructions are configured to, when executed by the processor, cause the wearable device to:
in case that the wearing position corresponds to the first position or the second position, maintain the measurement of oxygen saturation; and
in case that the wearing position corresponds to the third position, stop oxygen saturation measurement.

7. The wearable device of claim 1, wherein the instructions are configured to, when executed by the processor, cause the wearable device to:
acquire an amplitude of alternating current (AC) and an amplitude of direct current (DC) from a PPG signal acquired from the PPG sensor;
in case that the amplitude of the AC exceeds the amplitude of the DC, determine that a wearing state of the wearable device is appropriate; and
in case that the amplitude of the AC is equal to or less than the amplitude of the DC, determine that the wearing state of the wearable device is inappropriate.

8. The wearable device of claim 7, wherein the instructions are configured to, when executed by the processor, cause the wearable device to, responsive to a determination that the wearing state of the wearable device is appropriate, maintain the measurement of oxygen saturation.

9. The wearable device of claim 8, wherein the instructions are configured to, when executed by the processor, cause the wearable device to:
determine whether a measured oxygen saturation exceeds a reference value;
in case that the measured oxygen saturation exceeds the reference value, provide an oxygen saturation measurement value; and
in case that the measured oxygen saturation is equal to or less than the reference value, provide the oxygen saturation measurement value and a warning notification.

10. The wearable device of claim 7, wherein the instructions are configured to, when executed by the processor, cause the wearable device to, responsive to a determination that the wearing state of the wearable device is inappropriate, stop the measurement of oxygen saturation.

11. The wearable device of claim 10, wherein the instructions are configured to, when executed by the processor, cause the wearable device to:
in case that a number of stops of the measurement of oxygen saturation corresponds to a designated number of times, provide a first measurement guide; and
in case that the number of stops of the measurement of oxygen saturation does not correspond to the designated number of times, provide a second measurement guide.

12. The wearable device of claim 11, wherein the instructions are configured to, when executed by the processor, cause the wearable device to, after providing the first measurement guide or the second measurement guide, determine whether there is no motion of the wearable device.

13. The wearable device of claim 1, wherein the instructions are configured to, when executed by the processor, cause the wearable device to:
after the oxygen saturation measurement is requested, in case that a number of detections of the motion of the wearable device corresponds to a designated number of detections, provide a first guide; and
after the oxygen saturation measurement is requested, in case that a number of detections of the motion of the wearable device does not correspond to the designated number of detections, provide a second guide.

14. An operation method of a wearable device, the method comprising:
in response to a request for measurement of oxygen saturation, acquiring motion data from an inertial sensor included in the wearable device;
if it is determined, based on the motion data, that there is no motion of the wearable device, determining a wearing hand on which the wearable device is worn, based on the motion data;
detecting a tilt of the wearable device during the measurement of oxygen saturation by means of a photoplethysmography (PPG) sensor included in the wearable device;
classifying wearing positions of the wearable device, based on the wearing hand and the tilt; and
providing different user interfaces according to the wearing positions.

15. The method of claim 14, further comprising:
in case that the wearing hand corresponds to a left hand, applying a left-hand tilt detection algorithm;
determining whether an angle between a wrist direction of the wearable device and a ground exceeds a first reference value according to the left-hand tilt detection algorithm; and
classifying the wearing positions of the wearable device according to a result of the determination

16. The method of claim 14, further comprising:
in case that the wearing hand corresponds to a right hand, applying a right-hand tilt detection algorithm;
determining whether the angle between the wrist direction of the wearable device and the ground is less than a second reference value according to the right-hand tilt detection algorithm; and
classifying the wearing positions of the wearable device according to a result of the determination.

17. The method of claim 14, further comprising:
in case that a wearing position corresponds to a first position, providing a first user interface associated with the first position;
in case that the wearing position corresponds to a second position, providing a second user interface associated with the second position, and
in case that the wearing position corresponds to a third position, providing a third user interface associated with the third position,
wherein the first position corresponds to a case where an angle between the wrist direction of the wearable device and a ground having a first value exceeding a first reference value or less than a second reference value,
wherein the second position corresponds to a case where the angle between the wrist direction of the wearable device and the ground has a second value between the first reference value and the second reference value, and
wherein the third position corresponds to a case where the angle between the wrist direction of the wearable device and the ground has a third value opposite to the first position.

18. The method of claim 17, further comprising:
in case that the wearing position corresponds to the first position or the second position, maintaining oxygen saturation measurement and an operation of, and
in case that the wearing position corresponds to the third position, stopping oxygen saturation measurement.

19. The method of claim 14, further comprising:
acquiring an amplitude of alternating current (AC) and an amplitude of direct current (DC) from a PPG signal acquired from the PPG sensor;
in case that the amplitude of the AC exceeds the amplitude of the DC, determining that a wearing state of the wearable device is suitable; and
in case that the amplitude of the AC is equal to or less than the amplitude of the DC, determining that the wearing state of the wearable device is unsuitable.

20. The method of claim 14, further comprising:
when it is determined that the wearing state of the wearable device is suitable, maintaining the oxygen saturation measurement, an operation of determining whether the measured oxygen saturation exceeds a reference value;
in case that the measured oxygen saturation exceeds the reference value, providing an oxygen saturation measurement value;
in case that the measured oxygen saturation is equal to or less than the reference value, providing the oxygen saturation measurement value and a warning notification
when it is determined that the wearing state of the wearable device is unsuitable, stopping the oxygen saturation measurement;
in case that the number of oxygen saturation measurement stops corresponds to a designated number of times, providing a first measurement guide; and
in case that the number of oxygen saturation measurement stops does not correspond to the designated number of times, providing a second measurement guide.
